(19) United States
(12) Patent Application Publication
(10) Pub. No.: US 2017/0217911 A1
(43) Pub. Date: Aug. 3, 2017

(54) Process For Preparation Of Linezolid

(71) Applicant: Mankind Research Centre, Haryana (IN)

(72) Inventors: Sonu Kumar, Haryana (IN); Bhuwan Bhashkar, Haryana (IN); Anil Kumar, Haryana (IN)

(21) Appl. No.: 15/379,841

(22) Filed: Dec. 15, 2016

(30) Foreign Application Priority Data
Dec. 18, 2015 (IN) 4177/DEL/2015

(51) Int. Cl. C07D 263/20

(52) U.S. Cl. CPC C07D 263/20

(57) ABSTRACT

The present invention relates to an improved process for the preparation of Linezolid of Formula-I comprising reacting compound of Formula-II with compound of Formula-III in presence of metal base wherein, said metal base is prepared in situ in a single lot. The invention also relates to an isolated acetamide impurity of Formula-IV produced in the process for preparation of Linezolid, its purification and its use as a reference marker.

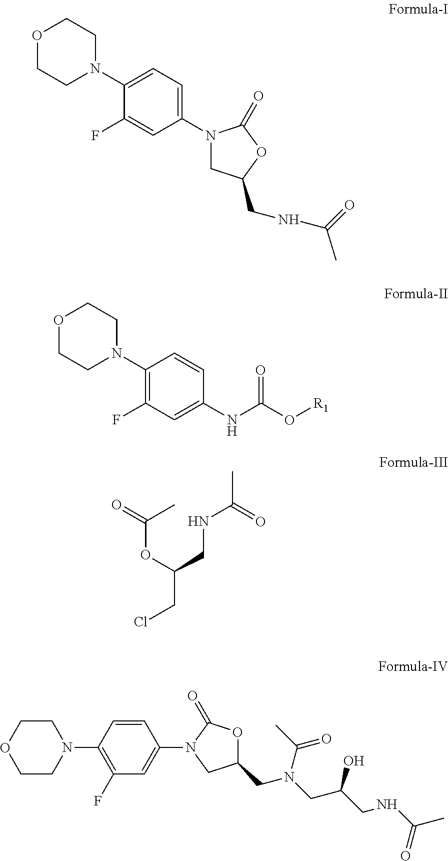

### FIELD OF THE INVENTION

The present invention relates to an improved, cost effective, commercially viable and industrially advantageous process for preparation of Linezolid of Formula-I in high yield. Linezolid obtained from the process of present invention is pure and substantially free from an acetamide impurity of Formula-IV.

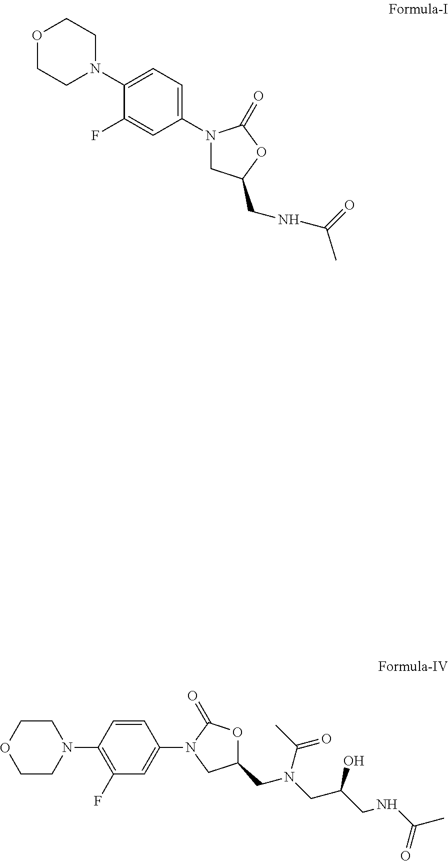 [SMILES: CC(=O)NC[C@H]1CN(c2ccc(N3CCOCC3)c(F)c2)C(=O)O1 ; CC(=O)NC[C@H](O)CN(C[C@H]1CN(c2ccc(N3CCOCC3)c(F)c2)C(=O)O1)C(C)=O]

### BACKGROUND AND OBJECT OF THE INVENTION

Linezolid, (S)—N-[[3-[3-Fluoro-4-(4-morpholinyl) phenyl]-2-oxo-5-oxazolidinyl]methyl]-acetamide as represented by Formula-I, is a synthetic antibacterial agent of the oxazolidinone class. Linezolid was first approved by USFDA in April 2000 under the brand name ZYVOX® and since then marketed by M/s Pfizer worldwide as Tablet, Oral Suspension and IV Injection for the treatment of infections caused by aerobic Gram-positive bacteria.

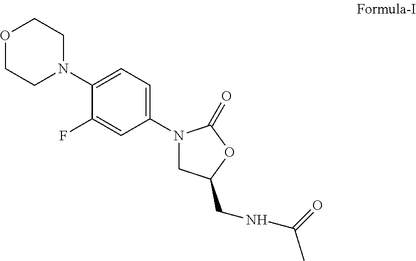 [SMILES: CC(=O)NC[C@H]1CN(c2ccc(N3CCOCC3)c(F)c2)C(=O)O1]

Linezolid was first disclosed in U.S. Pat. No. 5,688,792. U.S. Pat. No. 5,688,792 also discloses a process for preparation of Linezolid and related compounds.

The process for preparation of Linezolid reported in U.S. Pat. No. 5,688,792 is represented herein below as Scheme-1.

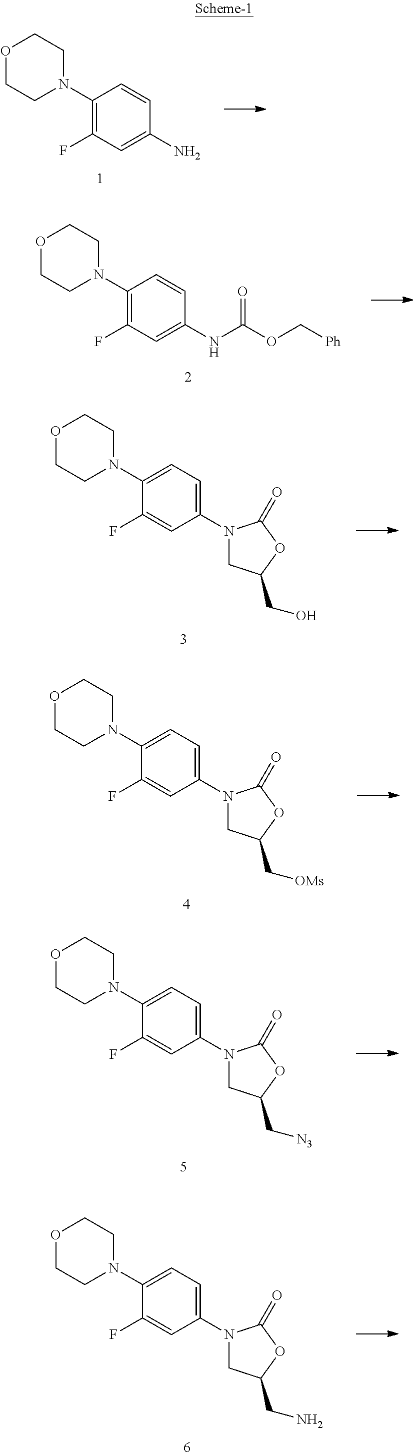 [SMILES: Nc1ccc(N2CCOCC2)c(F)c1 ; O=C(Nc1ccc(N2CCOCC2)c(F)c1)OCc1ccccc1 ; O=C1O[C@@H](CO)CN1c1ccc(N2CCOCC2)c(F)c1 ; CS(=O)(=O)OC[C@H]1CN(c2ccc(N3CCOCC3)c(F)c2)C(=O)O1 ; [N-]=[N+]=NC[C@H]1CN(c2ccc(N3CCOCC3)c(F)c2)C(=O)O1 ; NC[C@H]1CN(c2ccc(N3CCOCC3)c(F)c2)C(=O)O1]

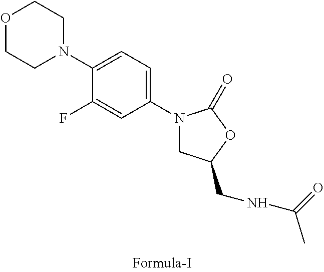 [SMILES: CC(=O)NC[C@H]1CN(c2ccc(N3CCOCC3)c(F)c2)C(=O)O1]

In the above synthesis, the intermediate azide, (R)—N-[[3-(3-fluoro-4-morpholinylphenyl)-2-oxo-5-oxazolidinyl]methyl]azide (5) is being prepared from (R)—N-[3-(3-fluoro-4-morpolinylphenyl)-2-oxo-5-oxazolidinyl]methyl methanesulfonate (4) using sodium azide as one of the critical reagents. The crude Linezolid so obtained is purified by column chromatography and the combined fractions are triturated with ethyl acetate to get pure Linezolid.

It is evident from US patent publication no. 2006/0252932 that when the said azide intermediate (5) is reduced to its corresponding amine, (S)—N-[[3-(3-fluoro-4-morpholinylphenyl)-2-oxo-5-oxazolidinyl]methyl]amine (6) in ethyl acetate by hydrogenation using hydrogen gas and a palladium/carbon catalyst, production of undesirable level of reaction by-products occur. The reaction is followed by acetylation of the intermediate amine (6) to Linezolid (Formula-I). In this process undesirably high level of bis-linezolid, as an impurity, is also obtained in final Linezolid product.

The major disadvantage of this process is the formation of high level of impurity, which makes the purification process very critical, resulting in poor yield of Linezolid. Purification techniques such as column chromatography is utilized to obtain pine Linezolid, which is time consuming and requires major volume of solvents making process tedious, expensive and not favorable for large scale production. Further, azide formation is not suitable for large scale production.

U.S. Pat. No. 7,087,784 discloses another route of synthesis of Linezolid, which involves condensation of N-carbobenzyloxy-3-fluoro-4-morpholin-4-yl aniline (2) with (S)—N-[2-(acetyloxy)-3-chloropropyl]-acetamide (Formula-III) in dimethyl formamide by addition of a base like lithium tertiarybutoxide-tetrahydrofuran solution as shown herein below in Scheme-2.

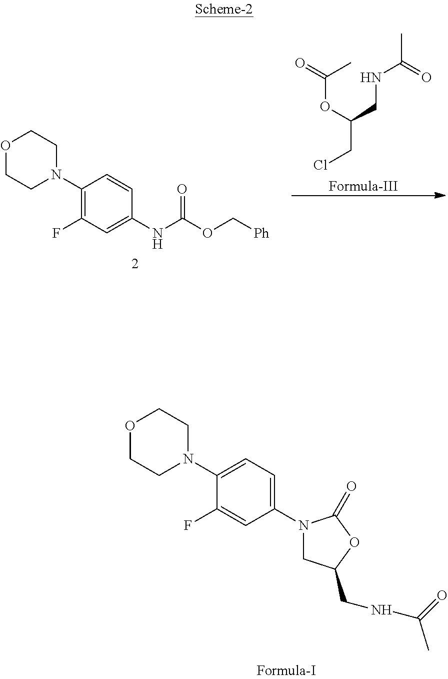

The above described synthesis has obvious advantages over Linezolid preparations disclosed in U.S. Pat. No. 5,688,792 and other related prior art methods, in such a way that it avoids the use of explosive sodium azide. However, lithium tertiarybutoxide-tetrahydrofuran solution used in such preparation as condensing agent is very expensive, explosive, difficult to transport, makes this process unsuitable at industrial production.

Similarly, Organic Process Research and Development, 7(4), 533-546, 2003; discloses a method of preparation of Linezolid of Formula-I by condensing compound (2) with compound of Formula-I in presence of a base like lithium tertiarybutoxide-tetrahydrofuran solution. The reaction conditions involve precarious parameters like cautious addition of lithium tertiarybutoxide-tetrahydrofuran solution under continuous flow of nitrogen to avoid its contact with atmospheric moisture.

It is known that lithium tertiarybutoxide-tetrahydrofuran solution is highly flammable and highly hygroscopic in nature. Costly handling and maintenance of lithium tertiarybutoxide-tetrahydrofuran solution make the above process uneconomical.

To avoid the above problems, Chinese patent publication No. 102206194 reported the use of a solid lithium-tertiarybutoxide base in a mixed solvent of dimethylformamide, methanol and dichloromethane as a condensing agent in place of lithium tertiarybutoxide-tetrahydrofuran solution in the similar condensation reaction as reported in U.S. Pat. No. 7,087,784. However, it is evident from another Chinese patent No. 103130733 that use of methanol in solvent system makes reaction exothermic, very intense and difficult to operate, which reduces the yield and makes this process unsuitable at industrial production.

Chinese patent No. 103130733 reported the use of solid lithium-tertiarybutoxide base in another solvent system as condensing agent, which avoids the use of methanol to overcome the above mentioned problem. The solvent system reported in CN 103130733 is a mixed solvent of n-propanol/iso-propanol/cyclohexanol/n-butanol and a dipolar solvent exemplified as N,N-dimethyl formamide, N,N-dimethyl acetamide and dimethyl sulfoxide.

The major drawback of above said process is cost incurred in storage and handling of solid lithium-tertiarybutoxide. The said base is highly hygroscopic at atmospheric conditions and requires critical parameters like nitrogen atmosphere and low temperature conditions for storage. It is so hygroscopic that it easily degrades even under small exposure to moisture and hence the reaction conditions disclosed by CN 103130733 had to be performed with precautions to avoid loss in yield. This makes process costly, unhandy and non-reproducible at large scale production.

None of the above mentioned prior arts offer simple and cost effective process for preparation of Linezolid.

The present invention is focused on the problems associated with the prior art processes and provides an improved process for preparation of Linezolid, wherein the process involves use of metal base like lithium-alkoxide which is prepared in situ, within the reaction vessel, to avoid the cost and labor incurred in its storage and handling.

Overall, the benefits of in situ prepared lithium-alkoxide as a condensing agent over lithium tertiarybutoxide-tetrahydrofuran solution and solid lithium-tertiarybutoxide base are as follows:
Transportation & handling of lithium metal and tetrahydrofuran separately is comparatively easy at industrial production;
Proper utilization of in situ prepared lithium-alkoxide as much required for one reaction batch makes the present process very economical, secondly in situ use of lithium-alkoxides, prevents its degradation by moisture and uncertainty of completion of reaction, increasing the overall yield of the product; and
In situ preparation of lithium-alkoxide in the reaction vessel as such does not require storage of unused lithium-alkoxide in moisture free environment eliminating the storage concern especially at large scale.

Hence, in situ preparation of required lithium-alkoxide as a condensing agent for one reaction batch is very economical in comparison to separately procured solid lithium tertiarybutoxide or lithium tertiarybutoxide-tetrahydrofuran solution, which makes the manufacturing process of present invention economical and practically feasible at large scale production in comparison to prior art processes.

Like any synthetic compound, Linezolid obtained by any process can contain unessential compounds or impurities coming from many sources such as from unreacted starting materials, by product of the reaction, products of side reactions or degradation products. It is essential to verify the identity of the source material and to establish its quality, otherwise impurities associated with the raw materials may be carried through the manufacturing process to contaminate the final product.

It is always desirable to prepare highly pure pharmaceutical products or a product having minimum amount of impurities which leads to negligible to reduce adverse side effects and to improve the shelf-life of active ingredient, as well as its formulation. Further, it is an essential requirement of every regulatory submission that the submissions are made to regulatory authorities along with the analytical data demonstrating the absence of impurities from the drug at the time of manufacture, or to demonstrate their presence only at a negligible level. These data are usually obtained by testing the drug against a reference, which is a suitably pure sample of a potential impurity.

Thus, the object of the present invention is to develop a cost effective, reproducible and industrial advantageous process for the preparation of Linezolid in high yield that must be free from any undesirable byproduct or impurity.

### SUMMARY OF THE INVENTION

In the first aspect, present invention relates to a process for preparation of Linezolid of Formula-I:

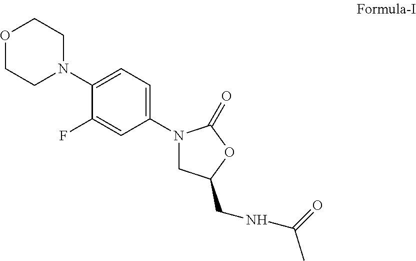 [SMILES: CC(=O)NC[C@H]1CN(c2ccc(N3CCOCC3)c(F)c2)C(=O)O1]

which comprises the steps of:
a) condensing a compound of Formula-II or its acid addition-salt

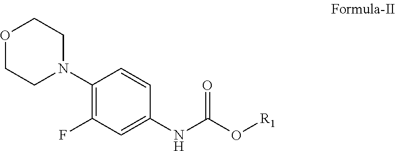

wherein, R₁ is selected from cycloalkyl, phenyl, —CH₂-phenyl, C₂₋₆ alkenyl, or C₁₋₆ alkyl optionally substituted by one to three atom(s) of F, Br, Cl, and —O—C₁₋₆ alkyl, with a compound of Formula-III

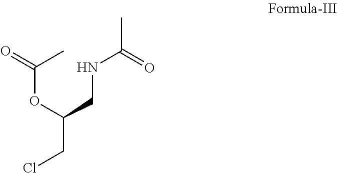

in presence of a metal base and a solvent to get a reaction mixture; wherein, said metal base is prepared in situ, in a single lot;
b) quenching the reaction mixture with aqueous ammonium chloride solution followed by extraction with dichloromethane to obtain a crude Linezolid;
c) crystallizing the crude Linezolid with a suitable solvent to obtain the Linezolid of Formula-I.

In second aspect, the present invention relates to an isolated acetamide impurity of Formula-IV.

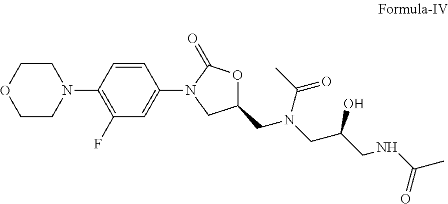 [SMILES: CC(=O)NC[C@H](O)CN(C[C@H]1CN(c2ccc(N3CCOCC3)c(F)c2)C(=O)O1)C(C)=O]

In third aspect, the present invention relates to a process for preparation of acetamide impurity of Formula-IV, comprises the steps of:
a) reacting a compound of Formula-V

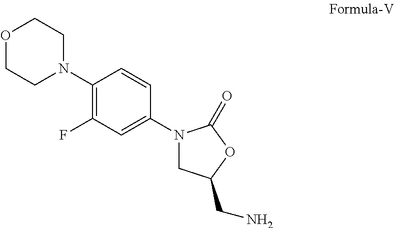 [SMILES: NC[C@H]1CN(c2ccc(N3CCOCC3)c(F)c2)C(=O)O1]

with a compound of Formula-II

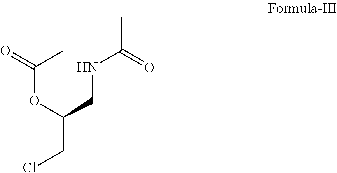

in presence of sodium hydride or potassium hydride, to obtain a compound of Formula-VI;

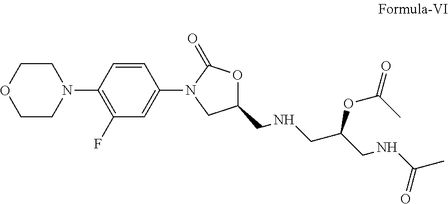 [SMILES: CC(=O)NC[C@@H](CNC[C@H]1CN(c2ccc(N3CCOCC3)c(F)c2)C(=O)O1)OC(C)=O]

b) treating the compound of Formula-VI with acetic anhydride to obtain a compound of Formula-VII;

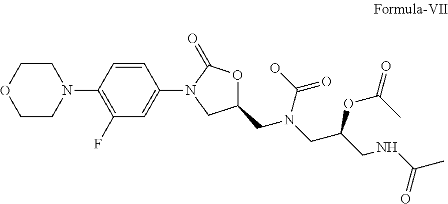 [SMILES: CC(=O)NC[C@@H](CN(C[C@H]1CN(c2ccc(N3CCOCC3)c(F)c2)C(=O)O1)C(=O)O)OC(C)=O]

c) hydrolyzing the compound of Formula-VII in presence of lithium hydroxide to obtain the compound of Formula-IV.

In fourth aspect, the present invention is related to a method for the quantification of the purity of Linezolid, comprising the use of acetamide impurity of Formula-IV as a reference standard.

The above and other objects of the present invention are further attained and supported by the following embodiments described herein. However, the described embodiments are in accordance with the best mode of practice and scope of the invention is not restricted to the described embodiments herein after.

### BRIEF DESCRIPTION OF THE FIGURES

FIG. 1 is an illustration of ¹³C NMR spectrum of acetamide impurity of Formula-IV

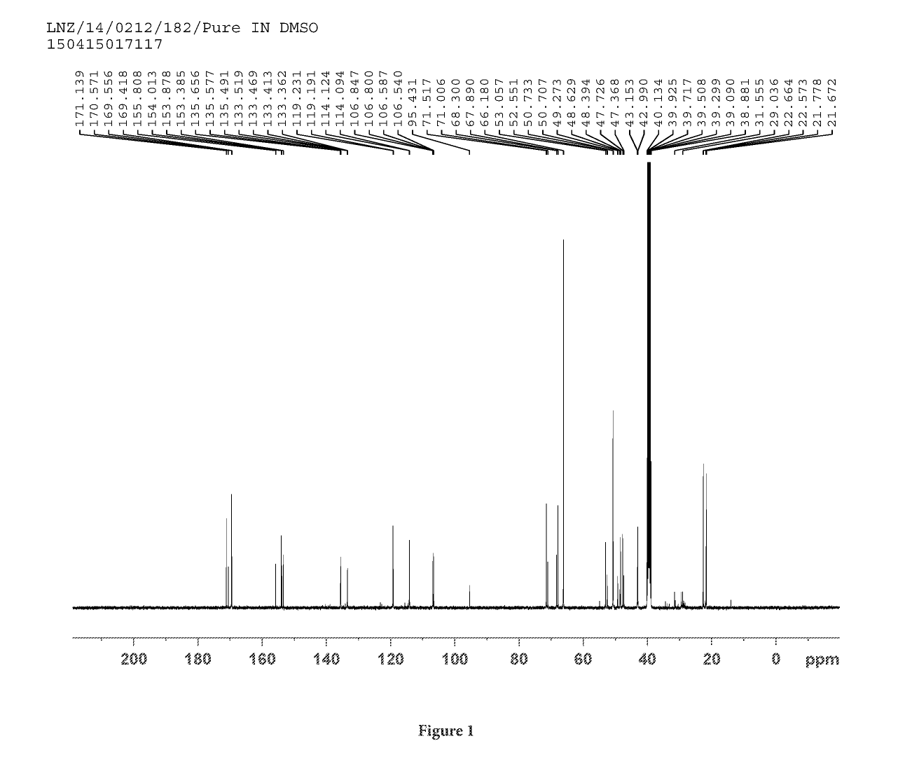

FIG. 2 is an illustration of ¹H NMR spectrum of acetamide impurity of Formula-IV

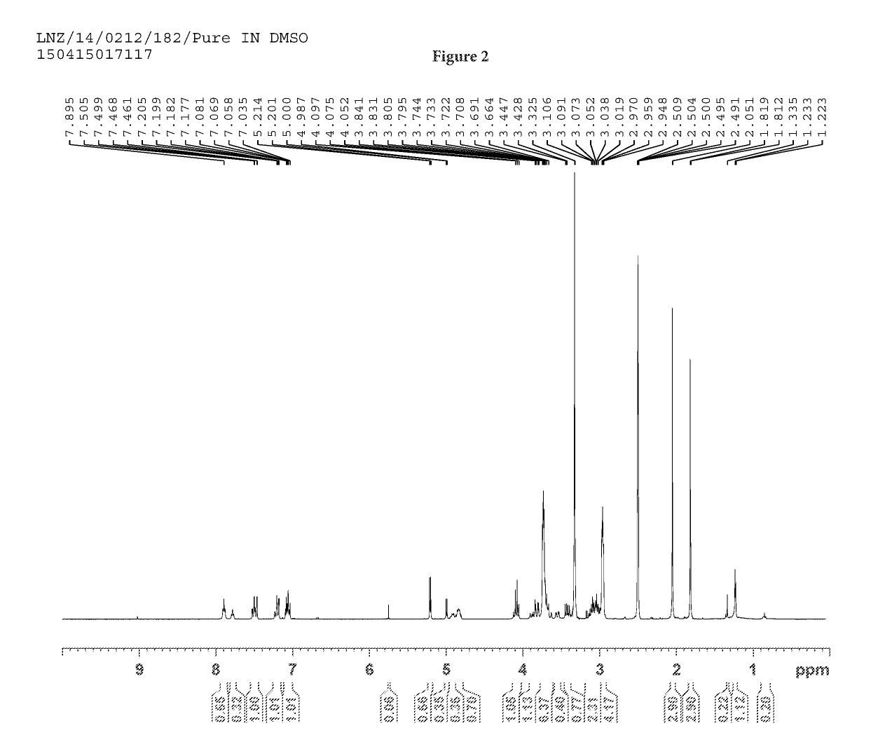

FIG. 3 is an illustration of FT-IR spectrum of acetamide impurity of Formula-IV

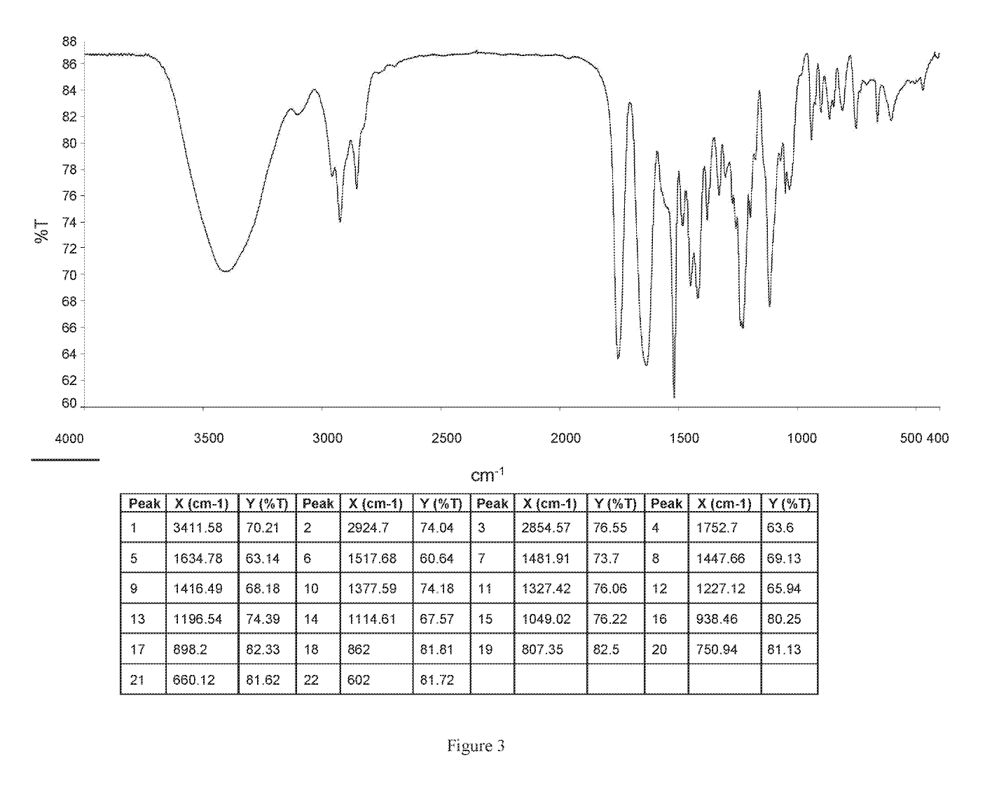

### DETAILED DESCRIPTION OF THE INVENTION

As used herein, the terms and phrases have the meanings, definitions, and explanations known in the art. Some of the more commonly used phrases are described in more detail below.

The term “in situ” means within the reaction wherein, the compounds formed in the reaction are taken to the next step as such, without isolation from the reaction vessel.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Alkyl refers to both straight and branched groups.

Alkenyl refers to both straight and branched alkyl groups containing one or more double bonds in the carbon chain.

Cycloalkyl refers to three to seven membered cycloalkyl ring system.

Alkoxy refers to a —O-alkyl group wherein alkyl is a straight or branched alkyl group.

Aryl refers to phenyl, pyridyl or naphthyl, which may be optionally substituted with one or more F, Cl, Br, I, CN, OH, SH, C₁₋₆ alkyl, OC₁₋₆ alkyl, or SC₁₋₆ alkyl, or —OC(O)CH₃.

Halogen or halo refers to fluorine, chlorine, bromine, and iodine.

HPLC refers to the well-known technique of high-performance liquid chromatography, also referred to as high pressure liquid chromatography. HPLC can be applied for detection and quantification of components of a mixture, for example detection and quantification of impurities in a principal compound such as an active pharmaceutical ingredient (API).

“FT-IR” refers to Fourier Transform Infra-Red Spectroscopy. FT-IR is a well-known spectroscopy analysis in which absorption of IR energy by the sample results from transitions between molecular vibrational energy levels. FT-IR is used, in modern practice, mainly for identification of functional groups in the molecule.

As used herein, the term ¹H NMR refers to proton nuclear magnetic resonance spectroscopy, ¹³C NMR refers to ¹³C nuclear magnetic resonance spectroscopy and LCMS refers to liquid chromatography-mass spectroscopy.

As used herein, the term “isolated” refers to a compound having purity of at least 90% (by HPLC).

As used herein “crude Linezolid” refers to Linezolid having purity≦99% (w/w), wherein the acetamide impurity of Formula-IV may be present in an amount≧0.50% (w/w).

As used herein “potency” refers to 100−(moisture content or LOD+residual solvents+heavy metals+sulphated ash+impurities).

Room temperature refers to an ambient temperature ranging between 25° C. to 30° C.

According to first aspect of the present invention, the process for preparation of Linezolid of Formula-I, comprises the following steps:
a) condensing a compound of Formula-II or its acid addition-salt

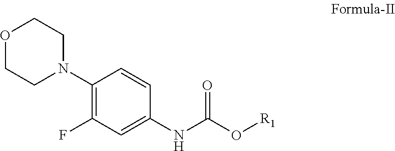

wherein, R₁ is selected from cycloalkyl, phenyl, —CH₂-phenyl, C₂₋₆ alkenyl, or C₁₋₆ alkyl optionally substituted by one to three atom(s) of F, Br, Cl, and —O—C₁₋₆ alkyl, with a compound of Formula-IM

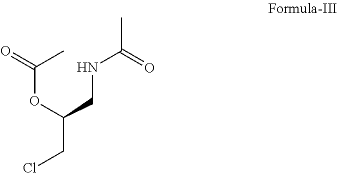

in presence of a metal base and a solvent; wherein, said metal base is prepared in situ, in a single lot.

In a preferred embodiment, the R₁ of compound of Formula-II is an ethyl group.

Compounds of Formula-II and Formula-III used in this step may be prepared as per the conventional methods reported in the prior arts.

The metal base in this step is prepared by reaction of metal with alcohol in ethereal solvent wherein, said metal is added in one lot to the ethereal solvent containing an alcohol.

The ethereal solvent may be selected from dioxane, diisopropyl ether, tetrahydrofuran and mixture thereof preferably, the ethereal solvent is tetrahydrofuran.

The metal employed in preparation of said metal base in step a) is lithium metal. The said metal base used in step a) is lithium-alkoxide.

Alcohol used for preparation of lithium-alkoxide may be selected from C₁-C₅ aliphatic alcohols such as isopropanol, isopentanol, isobutanol, textiatybutanol and the like, preferably the alcohol used is selected from tertiarybutanol and isopropanol.

Lithium-alkoxide base prepared within the reaction vessel, may be selected from lithium-tertiarybutoxide, lithium-isopropoxide and lithium-isopentoxide, preferably the lithium-alkoxide base is selected from lithium-tertiarybutoxide and lithium-isopropoxide.

Lithium metal used in the preparation of lithium-alkoxide in this step may be taken in an amount ranging from 2.0 to 3.5 mole equivalents with respect to compound of Formula-II, preferably 3.0 mole equivalent with respect to compound of Formula-II.

The reaction may be carried out at a temperature between 40° C. to 85° C., preferably the reaction is carried out at a temperature between 55° C. to 70° C.

In the condensation reaction, the compound of Formula-II may be taken in an amount between 1.0 to 3.0 mole equivalents with respect to compound of Formula-II, preferably 2 mole equivalent with respect to compound of Formula-II.

The condensation may be carried out in presence of a solvent selected from acetonitrile; N,N-dimethylformamide; N,N-dimethylacetamide, dimethylsulfoxide, tetrahydrofuran, isopropanol, tertiarybutanol and the mixture thereof. Preferably, the solvent is selected from N,N-dimethylacetamide, tetrahydrofuran, isopropanol, tertiary butanol and the mixture thereof.

The condensation may be carried out at a temperature between −5° C. to 35° C., preferably, the reaction is carried out between 0° C. to 30° C.

Further, steps b) and c) of the process according to the first aspect of present invention are defined here in below:
b) quenching the reaction mixture with aqueous ammonium chloride solution followed by extraction with dichloromethane to obtain crude Linezolid;
c) crystallizing crude Linezolid with suitable solvent to obtain Linezolid of Formula-I.

In one embodiment, the crude Linezolid obtained herein may be isolated from the reaction vessel and then subjected to crystallization.

In an alternative embodiment, the crude Linezolid obtained herein may not be isolated from the reaction vessel and obtained residue is then directly subjected to crystallization.

In one embodiment, crystallization of crude Linezolid is carried out by heating crude Linezolid in a solvent or a mixture of solvents up to reflux, and then cooling to 0° C.

The solvent used in crystallization of crude Linezolid may be selected from dichloromethane, toluene, acetone, acetonitrile, ethyl acetate, ethanol, methanol, isopropanol, n-propanol and mixture thereof. Preferably, the solvent is selected from ethyl acetate and isopropanol.

The crystallization of crude Linezolid may be carried out at temperature ranging from 5° C. to 100° C.

In one embodiment, the starting material ethyl (3-fluoro-4-morpholinophenyl)carbamate of Formula-II (R₁ is ethyl) may be recovered from mother liquor which is obtained after crystallization of Linezolid in ethyl acetate.

In a preferred embodiment, the Linezolid obtained according to the process of present invention is substantially free from the acetamide impurity of Formula-IV.

As used herein, “substantially free” refers to Linezolid having purity>99% (w/w), wherein, the acetamide impurity of Formula-IV may be present in an amount<0.50% (w/w), preferably may be present in an amount<0.20%, most preferably, may be present in an amount<0.10% (w/w).

According to second aspect of the present invention, there is provided an isolated acetamide impurity of Formula-IV having relative retention time (RRT) of 1.16 as measured by HPLC according to the method described in Indian Pharmacopoeia.

The acetamide impurity of Formula-IV is characterized by ¹H NMR, ¹³C NMR, FT-IR and LCMS.

Acetamide impurity of Formula-IV may be isolated from the mother liquor which is obtained after crystallization of crude Linezolid according to the process of present invention.

In one embodiment, the said mother liquor is concentrated under vacuum at 50° C. to get the residue which is purified by column chromatography.

Acetamide impurity of Formula-IV may be eluted in mixture of ethyl acetate and methanol, then desired fractions are collected and concentrated at 5° C. to obtain pure acetamide impurity of Formula-IV.

In one embodiment, the isolated acetamide impurity of Formula-IV may have purity more than 90% as measured by HPLC.

To the best of applicants' knowledge, the structure of acetamide impurity of Formula-IV has never been discovered before. This impurity may be formed during the condensation reaction of compounds of Formula-II and Formula-III in the process of preparation of crude Linezolid of present invention.

According to third aspect of the present invention, the process for preparation of acetamide impurity of Formula-IV, comprises the following steps:
a) reacting a compound of Formula-V

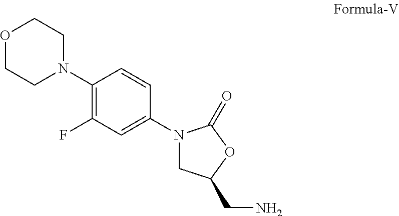 [SMILES: NC[C@H]1CN(c2ccc(N3CCOCC3)c(F)c2)C(=O)O1]

with a compound of Formula-I

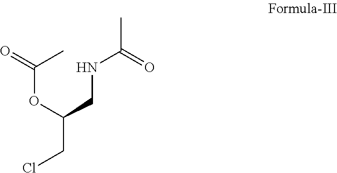

in presence of sodium hydride or potassium hydride, to obtain a compound of Formula-VI;

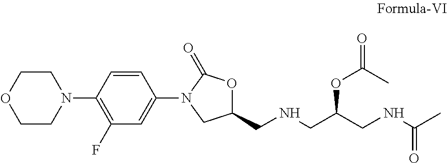 [SMILES: CC(=O)NC[C@@H](CNC[C@H]1CN(c2ccc(N3CCOCC3)c(F)c2)C(=O)O1)OC(C)=O]

b) treating the compound of Formula-VI with acetic anhydride to obtain a compound of Formula-VII;

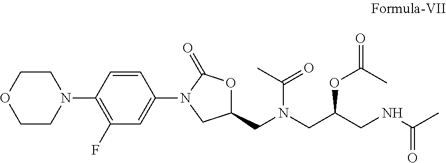 [SMILES: CC(=O)NC[C@@H](CN(C[C@H]1CN(c2ccc(N3CCOCC3)c(F)c2)C(=O)O1)C(C)=O)OC(C)=O]

c) hydrolyzing the compound of Formula-VII in presence of lithium hydroxide to obtain the compound of Formula-IV.

In one embodiment the acetamide impurity of Formula-IV has utility as a reference marker for Linezolid because it is a potential contaminant arising from side reactions which occur during the synthesis of Linezolid.

In another embodiment the test sample of Linezolid to be analyzed is assayed by one or more conventional analytical techniques. The analytical technique includes high performance liquid chromatography (HPLC) which is used for detection and quantification of impurities in a principal compound such as the drug substance. Detection and especially quantification of components of a mixture can be accomplished with the use of response factors. The response of a detector in HPLC (e.g. UV detectors or refractive index detectors) can be different for each compound eluting from the HPLC column. Response factors, as known, account for this difference in the response signal of the detector to different compounds eluting from the column.

### Analytical Methods:

Acetamide impurity of Formula-IV may be determined using HPLC methods as per given in Indian pharmacopoeia.

Typical retention time

| | | | Relative |
|---|---|---|---|
| | Retention | Relative retention | response factor |
| Peak Name | time (min.) | time (RRT) | (RRF) |
| Linezolid | 16.2 | 1.0 | — |
| Acetamide | 18.7 | 1.16 | 0.67 |
| impurity | | | |

The percentage of each identified and isolated impurity is calculated using following Formula-:

$$Impurity ( w / w \% ) = \frac{\begin{matrix}Area of impurity in test sample \times wt . of standard of Linezolid \times \\ dilution of test sample \times potency of standard of Lenezolid\end{matrix}}{\begin{matrix}Area of Standard of Linezolid \times wt . of test sample \times \\ dilution of standard of Linezolid \times R R F of Acetamide impurity\end{matrix}}$$

It is observed that response of acetamide impurity is less than Linezolid, so on the basis of RRF of acetamide impurity, i.e. 0.67 the actual w/w % of acetamide impurity present in Linezolid samples may be calculated using abovementioned formula.

Further, the present invention is illustrated in detail by way of the following examples. The examples are given herein for illustration of the invention and are not intended to be limiting thereof.

### EXAMPLES

### Example 1

### Preparation of Linezolid within Situ Synthesis of Lithium Tertiarybutoxide

In a round bottom flask, charged lithium metal (1.0 g, 0.144 moles), tetrahydrofuran (50 ml) and tertiarybutanol (32.17 g, 0.434 moles) and heated reaction mass to 55-70° C. Distilled out the solvent and cooled the residue to room temperature. Added N,N-dimethyl acetamide (20.0 ml) and cooled to 0° C. to 10° C. Added a solution of ethyl (3-fluoro-4-morpholinophenyl)carbamate (12.3 g, 0.0458 moles) of Formula-II and (S)-1-acetamido-3-chloropropan-2-yl acetate (18.0 g, 0.093 moles) of Formula-II and isopropanol (7 ml) in N,N-dimethyl acetamide at 0° C. Stirred the reaction mass at room temperature. Monitor the progress of reaction by HPLC. Then cooled the reaction mass to 0° C. and quenched with aqueous NH₄Cl solution and extracted with dichloromethane. Organic layer was washed with water and concentrated under reduced pressure to get crude Linezolid.

HPLC purity: 90.47% (w/w)

Acetamide impurity: 1.4% (w/w)

### Purification:

Crude Linezolid as a residue was crystallized with ethyl acetate without isolating from reaction vessel. The solid obtained was filtered, washed with ethyl acetate and dried. Solid material (14 g) thus obtained was further re-crystallized in isopropanol. The solid obtained was filtered, washed with isopropanol and dried under vacuum to get pure Linezolid (13.4 g).

From mother liquor obtained after crystallization of Linezolid in ethyl acetate, starting material ethyl (3-fluoro-4-morpholinophenyl)carbamate was recovered by crystallization in acetone:water, 0.5 g.

Yield: 13.4 g, 86.64% (w/w)

Percentage yield on the basis of consumed starting material: 90.3% (w/w)

HPLC purity: 99.94% (w/w)

Acetamide impurity: 0.05% (w/w)

### Example 2

### Preparation of Linezolid within Situ Synthesis of Lithium Iso-Propoxide

In a round bottom flask charged lithium metal (0.78 g, 0.113 moles), tetrahydrofuran (50 ml) and isopropanol (20 ml) and heated reaction mass to 55-70° C. Distilled out solvent and cooled the residue to room temperature. Added N,N-dimethylacetamide (18 ml) and cooled to 0° C. to 10° C. Added a solution of ethyl (3-fluoro-4-morpholinophenyl)carbamate (10 g, 0.0373 moles) of Formula-II and (S)-1-acetamido-3-chloropropan-2-yl acetate (15.0 g, 0.077 moles) of Formula-II in N,N-dimethylacetamide at 0° C. Stirred the reaction mass at room temperature. Monitor the progress of reaction by HPLC. Then cooled the reaction mass to 0° C. and quenched with aqueous NH₄Cl solution and extracted with dichloromethane. Organic layer was washed with water and concentrated under reduced pressure to get crude material.

HPLC Purity: 83.34% (w/w)

Acetamide impurity: 1.18% (w/w)

### Purification:

Crude Linezolid as a residue was crystallized with ethyl acetate without isolating from reaction vessel. The solid obtained was filtered, washed with ethyl acetate and dried. Solid material (10.7 g) thus obtained was further re-crystallized in isopropanol. The solid obtained was filtered, washed with isopropanol and dried under vacuum to get pure Linezolid (9.94 g).

From mother liquor obtained after crystallization of Linezolid in ethyl acetate, starting material ethyl (3-fluoro-4-morpholinophenyl)carbamate was recovered by crystallization as in Example-1 to give 0.7 g starting material.

Yield: 9.94 g, 79.05% (w/w)

Percentage yield on the basis of consumed starting material: 85.0% (w/w)

HPLC purity: 99.94% (w/w)

Acetamide impurity: 0.04% (w/w)

### Example 3

### Isolation of acetamide impurity, viz. N—((S)-3-acetamido-2-hydroxypropyl)-N—(((S)-3-(3-fluoro-4-morpholinophenyl)-2-oxooxazolidin-5-yl)methyl)acetamide of Formula-IV

Mother liquor obtained after crystallization of purified Linezolid in isopropanol was concentrated under vacuum at 50° C. to get residue. This residue was purified by column chromatography using silica gel. The desired compound was eluted in ethyl acetate:methanol and desired fractions were collected and concentrated on rotavapour at 50° C. under vacuum to get pure material of HPLC purity: 91.7%

### Example 4

### Synthesis of acetamide impurity, viz. N—((S)-3-acetamido-2-hydroxypropyl)-N—(((S)-3-(3-fluoro-4-morpholinophenyl)-2-oxooxazolidin-5-yl)methyl)acetamide of Formula-IV

### Step-a): Synthesis of (R)-1-acetamido-3-((((S)-3-(3-fluoro-4-morpholinophenyl)-2-oxooxazolidin-5-yl)methyl)amino)propan-2-yl acetate represented by Formula-VI

S—N-[(3-(3-fluoro-4-[4-morpholinyl]phenyl]-2-oxo-5-oxazolidinyl]methylamine (2 g, 0.0067 moles) of Formula-V was dissolved in 15 ml DMF and cooled to 0° C. Added sodium hydride (60% dispersion in mineral oil) (0.542 g, 0.0135 moles) at 0° C. Added (S)-1-acetamido-3-chloropropan-2-yl acetate (1.96 g, 0.0101 moles) of Formula-III. Reaction mixture was stirred at room temperature for 2 h. Reaction mixture was cooled to 0° C. and quenched with 1 ml acetic acid and 10% K₂CO₃ aqueous solution. The compound was extracted with dichloromethane and organic layer was washed with water and concentrated to obtain (R)-1-acetamido-3-((((S)-3-(3-fluoro-4-morpholinophenyl)-2-oxooxazolidin-5-yl)methyl)amino)propan-2-yl acetate of Formula-VI.

Yield: 3 g, 97.9% (w/w)

### Step-b): Synthesis of (S)-1-acetamido-3-(N—(((S)-3-(3-fluoro-4-morpholinophenyl)-2-oxooxazolidin-5-yl)methyl)acetamido)propan-2-yl acetate represented by Formula-VII

In a round bottom flask, a solution of (R)-1-acetamido-3-((((S)-3-(3-fluoro-4-morpholinophenyl)-2-oxooxazolidin-5-yl)methyl)amino)propan-2-yl acetate (3 g, 0.0066 moles) of Formula-VI in dichloromethane was added at room temperature followed by addition of triethylamine (3.69 ml, 0.026 moles). Reaction mass was cooled to 0° C. and acetic anhydride (4.06 g, 0.0398 moles) was added. The reaction mixture was stirred at room temperature for 1 h and then quenched with water. The compound was extracted with dichloromethane and washed with water. The organic layer was concentrated to obtain crude material. The crude material was purified by column chromatography using silica gel. The desired product was eluted in ethyl acetate:methanol and pure fractions were collected and concentrated to get (S)-1-acetamido-3-(N—(((S)-3-(3-fluoro-4-morpholinophenyl)-2-oxooxazolidin-5-yl)methyl)acetamido)propan-2-yl acetate of Formula-VII.

Yield: 1.2 g, 36.6% (w/w)

### Step-c): Synthesis of N—((S)-3-acetamido-2-hydroxypropyl)-N—(((S)-3-(3-fluoro-4-morpholinophenyl)-2-oxooxazolidin-5-yl)methyl)acetamide of Formula-IV

In a round bottom flask a solution of (S)-1-acetamido-3-(N—(((S)-3-(3-fluoro-4-morpholinophenyl)-2-oxooxazolidin-5-yl)methyl)acetamido)propan-2-yl acetate (1.2 g, 0.0024 moles) in tetrahydrofuran and methanol was added at room temperature. Cool the reaction mass to 0° C. Added 3 ml water and lithium hydroxide (0.408 g, 0.0097 moles) in reaction mass and stirred for 30 minutes. The compound was extracted with dichloromethane and washed with water. The organic layer was concentrated to obtain crude material. The crude material was purified by column chromatography using silica gel. The desired product was eluted in dichloromethane:methanol and pure fractions were collected and concentrated to get N—((S)-3-acetamido-2-hydroxypropyl)-N—(((S)-3-(3-fluoro-4-morpholinophenyl)-2-oxooxazolidin-5-yl)methyl)acetamide of Formula-IV.

Yield: 0.5 g, 45.8% (w/w)

Potency: 98.50%

### Characterization Data of Acetamide Impurity of Formula-IV:

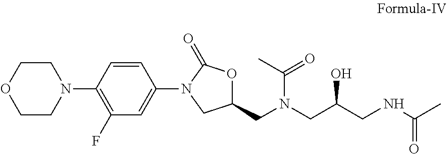 [SMILES: CC(=O)NC[C@H](O)CN(C[C@H]1CN(c2ccc(N3CCOCC3)c(F)c2)C(=O)O1)C(C)=O]

1. ¹³C NMR (In DMSO)—

Instrumentation: Bruker Asend 400 at 400 MHz

171.139, 170.571, 169.556, 169.418, 155.808, 154.878, 154.013, 153.878, 153.385, 135.577, 135.656, 135.491, 133.519, 133.469, 133.413, 133.362, 119.231, 119.191, 114.124, 114.094, 106.847, 106.800, 106.587, 106.540, 71.517, 71.006, 68.300, 67.890, 66.180, 53.057, 52.551, 50.733, 49.273, 48.629, 48.394, 47.726, 47.368, 43.153, 42.990, 22.664, 22.573, 21.778, 21.672.

2. ¹H NMR (In DMSO)—

Instrumentation: Bruker Asend 400 at 400 MHz

7.91-7.78 (m, 1H), 7.52-7.46 (m, 1H), 7.23-7.17 (m, 1H), 7.09-7.035 (m, 1H), 5.21-4.98 (m, 1H), 4.85-4.82 (m, 1H), 4.09-4.05 (m, 1H), 3.84-3.74 (m, 1H), 3.73-3.66 (m, 7H), 3.44-3.325 (m, 2H), 3.12-2.94 (m, 6H), 2.051 (S, 3H), 1.817-1.812 (d, 3H).

3. FT-IR Spectroscopy—

Instrumentation: PerkinElmer FT-IR Spectrophotometer (Spectrum-two).

3411.5 cm⁻¹, 2924.7 cm⁻¹, 2854.57 cm⁻¹, 1752.7 cm⁻¹, 1634.78 cm⁻¹, 1517.68 cm⁻¹, 1481.9 cm⁻¹, 1447.6 cm⁻¹, 1416.49 cm⁻¹, 1377.59 cm⁻¹, 1327.42 cm⁻¹, 1227.1 cm⁻¹, 1196.5 cm⁻¹, 1114.6 cm⁻¹, 1049.02 cm⁻¹, 938.5 cm⁻¹, 898.2 cm⁻¹, 862 cm⁻¹, 807.35 cm⁻¹, 750.9 cm⁻¹, 660.12 cm⁻¹, 602 cm⁻¹

4. LCMS:

Instrumentation and sample preparation: Waters Xevo TQD

(M+)=453.24

We claim:

1. A process for preparation of Linezolid of Formula-I; 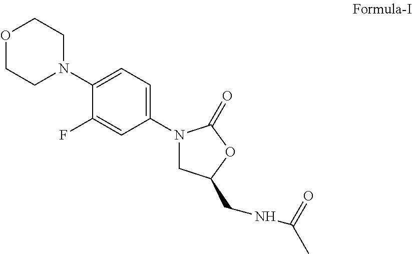 [SMILES: CC(=O)NC[C@H]1CN(c2ccc(N3CCOCC3)c(F)c2)C(=O)O1]
comprises the steps of:
a) condensing a compound of Formula-II or its acid addition-salt 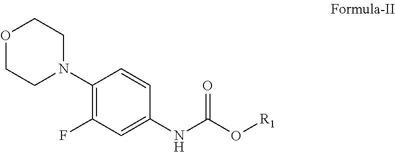
wherein, R₁ is selected from cycloalkyl, phenyl, —CH₂-phenyl, C₂₋₆ alkenyl, or C₁₋₆ alkyl optionally substituted by one to three atom(s) of F, Br, Cl, and —O—C₁₋₆ alkyl, with a compound of Formula-III 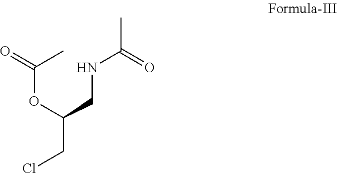
in presence of a metal base and a solvent to get a reaction mixture, wherein, said metal base is prepared in situ, in a single lot;
b) quenching the reaction mixture with aqueous ammonium chloride solution followed by extraction with dichloromethane to obtain a crude Linezolid;
c) crystallizing the crude Linezolid with a suitable solvents to obtain the Linezolid of Formula-I.

2. The process according to claim 1, wherein in step a), said metal base is prepared by addition of a metal in single portion to an ethereal solvent containing alcohol at room temperature and heating to temperature ranging from 40° C. to 85° C.

3. The process according to claim 2, wherein said metal is lithium.

4. The process according to claim 2, wherein said ethereal solvent is selected from tetrahydrofuran, diisopropyl ether, dioxane or mixture thereof.

5. The process according to claim 2, wherein said alcohol is selected from C₁-C₅ aliphatic alcohols.

6. The process according to claim 1, wherein said metal base is lithium-alkoxide.

7. The process according to claim 6, wherein said lithium-alkoxide is selected from lithium tertiarybutoxide and lithium isopropoxide.

8. The process according to claim 1, wherein R₁ is ethyl (—CH₂—CH₃).

9. The process according to claim 1, wherein in step a), said solvent is selected from acetonitrile, N,N-dimethylformamide, N,N-dimethylacetamide, dimethylsulfoxide, tetrahydrofuran, isopropanol, tertiary butanol and the mixture thereof.

10. The process according to claim 1, wherein in step c), said crystallization is carried out by heating the crude linezolid in the solvents at a temperature ranging from room temperature to reflux, and then cooling to 0° C.

11. The process according to claim 1, wherein in step c), said solvents are selected from dichloromethane, toluene, acetone, acetonitrile, ethyl acetate, ethanol, methanol, isopropanol, n-propanol and mixture thereof.

12. The process according to claim 1, wherein said Linezolid obtained in step c) is substantially free from acetamide impurity of Formula-IV

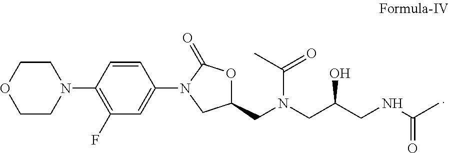 [SMILES: CC(=O)NC[C@H](O)CN(C[C@H]1CN(c2ccc(N3CCOCC3)c(F)c2)C(=O)O1)C(C)=O]

13. An isolated acetamide impurity of Formula-IV having relative retention time (RRT) of 1.16 as measured by HPLC

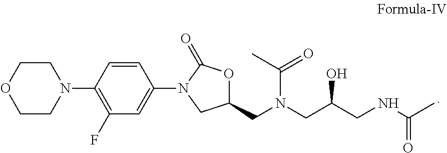 [SMILES: CC(=O)NC[C@H](O)CN(C[C@H]1CN(c2ccc(N3CCOCC3)c(F)c2)C(=O)O1)C(C)=O]

14. A process for preparing acetamide impurity of Formula-IV 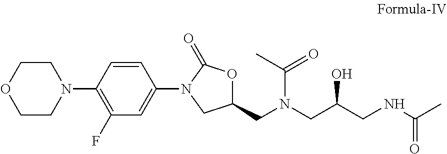 [SMILES: CC(=O)NC[C@H](O)CN(C[C@H]1CN(c2ccc(N3CCOCC3)c(F)c2)C(=O)O1)C(C)=O]
comprising the steps of:
a) reacting a compound of Formula-V 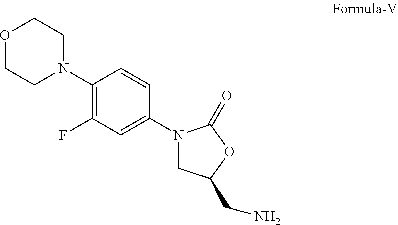 [SMILES: NC[C@H]1CN(c2ccc(N3CCOCC3)c(F)c2)C(=O)O1]
with a compound of Formula-III 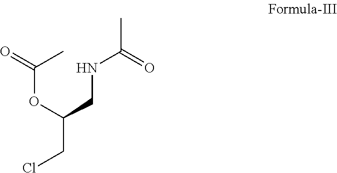
in presence of sodium hydride or potassium hydride, to obtain a compound of Formula-VI, 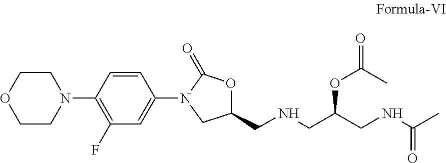 [SMILES: CC(=O)NC[C@@H](CNC[C@H]1CN(c2ccc(N3CCOCC3)c(F)c2)C(=O)O1)OC(C)=O]
b) treating the compound of Formula-VI with acetic anhydride to obtain a compound of Formula-VII; 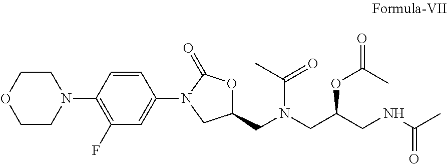 [SMILES: CC(=O)NC[C@@H](CN(C[C@H]1CN(c2ccc(N3CCOCC3)c(F)c2)C(=O)O1)C(C)=O)OC(C)=O]
c) hydrolyzing the compound of Formula-VII in presence of lithium hydroxide to obtain the compound of Formula-IV.
